(19) United States
(12) Patent Application Publication
(10) Pub. No.: US 2025/0186764 A1
(43) Pub. Date: Jun. 12, 2025

(54) ELECTRICAL LEAD ASSEMBLY FOR IN VIVO INSERTION INTO A BODY PASSAGE AND INCLUDING A FEATURE WHICH FACILITATES EXTRACTION OF A LEAD ASSEMBLY

(71) Applicant: GRJ Med Tech, LLC, Pepper Pike, OH (US)

(72) Inventor: Majid Rashidi, Pepper Pike, OH (US)

(73) Assignee: GRJ Med Tech, LLC, Pepper Pike, OH (US)

(21) Appl. No.: 18/972,777

(22) Filed: Dec. 6, 2024

Related U.S. Application Data
(60) Provisional application No. 63/607,307, filed on Dec. 7, 2023.

(51) Int. Cl. A61N 1/05

(52) U.S. Cl. CPC A61N 1/056

(57) ABSTRACT

A lead assembly for insertion into a body passage such as the lumen of a vein is particularly applicable for assembly of a cardiac pacemaker. In one version the lead has a distal end with an end portion of a flexible sheath attached with the sheath initially extending outwardly from the distal end and subsequently folded in an annular U-fold over the lead to provide a sheath along the length. By applying tensile force to the proximal end of the lead, the annular fold causes the material of the sheath to peel away from any surrounding scar tissue as the lead is pulled out towards the proximal end and provides for easy nondestructive extraction of the lead. In another version, a single layer flexible sheath is attached at the distal end of a lead, and, upon application of a tensile force to the stretchable sheath, the lead can be extracted.

### CROSS-REFERENCE TO RELATED APPLICATION

The present application claims priority from and benefit of the filing date of U.S. provisional patent application Ser. No. 63/607,307 filed Dec. 7, 2023, and the entire disclosure of said provisional application is hereby expressly incorporated by reference into the present application.

### BACKGROUND

The present disclosure relates to an electrical lead assembly for insertion into a body passage such as a vein and more particularly relates for example to an electrical lead assembly for connection of a subcutaneous pacemaker with the heart for providing electrical pulses to stimulate continued beating of the heart.

In service, after a sufficient period of time, it has been observed that infection can occur, or failure of the electrical wires of the lead connection to the cardiac muscle may occur, and thus requires extraction and replacement of the pacemaker lead assembly. However, neointimal fibrotic or scar tissue typically has formed naturally by the body about or around the lead, resulting in lead encapsulation and has retained the lead against the lumen wall of the vein. In current surgical procedures a rotary cutter has been employed over the lead to separate the lead from the surrounding scar tissue; however, the cutter has the undesired potential to damage or rupture the vein wall which can result in internal bleeding and associated undesired consequences.

Referring to FIG. 1, a portion of a prior art pacemaker installation or lead 4 is shown that is received through a passage such as vein 2 with blood flow through the lumen 3 of vein 2. The lead 4 has an electrical conductor 5 extending from a first or distal end thereof which provides a pacing signal to the heart 1. A second or proximal end of the lead 4 is connected to an electrical signal generator 6 or pacemaker which provides the electrical pulses to the interior of the heart to facilitate continued beating of the heart.

A known solution to this problem has been described in U.S. Pat. No. 10,933,247 which is expressly incorporated herein by reference in its entirety and describes a double layered sheath received over the lead where the inner layer is connected to the outer layer by an annular fold at the distal or end adjacent to the heart. Extraction of this known lead assembly is performed by applying a tensile force to the inner layer of the sheath at the proximal end adjacent to the electric pulse generating assembly. Continuous application of the tensile force causes the outer layer of the sheath to peel away progressively by movement of the annular fold from the distal end towards the proximal end, thus freeing the lead from the scar tissue and enabling withdrawal of the lead assembly. However, the solution of the aforesaid '247 patent uses a frangible seal about the annular fold at the distal end of the double layer sheath in order to prevent seepage or passage of blood between the sheath and the lead. This causes difficulty of assembly of the sheath over the lead and thus provides an extra level of complication in the manufacture of the sheath assembly. Thus, it has been desired to find a safe and simpler way of facilitating extraction of a cardiac pacemaker lead, where required, and a technique which readily lends itself to the original production of the cardiac pacemaker lead.

A need exists for an improved arrangement that addresses at least one or more of the above-described disadvantages, as well as still other features and benefits.

### SUMMARY

The present disclosure provides an improved arrangement of a sheath used in connection with a cardiac pacemaker lead.

The present arrangement provides a sheath for assembly over a cardiac pacemaker lead which utilizes a single layer or tube of a material disposed over the lead upon original manufacture of the pacemaker lead assembly and, one which is formed in a simplified manner and facilitates the assembly in vivo in the vein or lumen of the vein. Where required, extraction of the lead assembly is accomplished in a manner which peels away the sheath from any scar tissue which has formed about the sheath and thus enables the lead to be readily and easily extracted from the vein.

In one embodiment the sheath is formed by securing an end of a flexible tube over the lead by a suitable expedience such as weldment, adhesive bonding, or fasteners. The tube is then inverted and folded over the portion secured to the pacemaker lead. The free end of the tube, opposite the secured end, is drawn and disposed back over the secured portion to the proximal end of the lead adjacent to the pacemaker or signal generator. Upon proceeding to separate the lead from the scar tissue and extract the lead from the vein, a light tensile force is applied to the proximal end of the lead and the lead is moved or pulled from the distal end of the sheath, resulting in a movement of the annular fold provided at the distal end of the sheath towards the proximal end and progressive separation or peeling of the outer wall of the sheath from the scar tissue thereby freeing the lead for easy extraction. This embodiment thus requires only a single layer of tube, initially longer than required, to be secured to the distal end of the lead and folded back over itself forming an annular fold which is subsequently pulled and separated or peeled away from any scar tissue formed over its outer surface of the sheath.

In another version of the disclosure presented herein, one end of a single layer tubular sheath is disposed over the pacemaker lead distal end and secured to the lead in a suitable manner such as by weldment, adhesive bonding, fasteners, or the like. When it is desired to remove the lead, a slight tensile force is applied to the proximal end of the sheath such that the sheath is stretched. By Poisson's effect the region of the stretched sheath adjacent the nearest tissue is reduced in diameter resulting in separation or peeling of the sheath from the scar tissue formed thereover, starting at the proximal end. As stretching is continued, the separation or peeling propagates toward the distal end of the sheath thus freeing the sheath from the scar tissue and enabling the sheath to be easily removed from the vein and scar tissue. This embodiment thus provides a simpler manner of installation of the sheath over the lead and provides a simplified and lowcost manner of manufacturing a cardiac pacemaker lead assembly which can be readily removed where the circumstances require.

The disclosure includes the description of a method forming a first version of the sheath over the lead employing a semi-rigid tube temporarily sleeved over the lead up to a point of the end of the sheath. The material of the sheath is then folded back over the end of the semi-rigid tube. The tube with material folded thereon is retracted or moved from the distal end of the sheath material towards the proximal end of the lead, thus forming the sheath in a position with the portion thereof secured to the lead connected to the remainder of the sheath by an annular fold and the semi-rigid tube employed as a tool and then is removed.

Still other benefits and advantages of the present disclosure will become more apparent from reading and understanding the following detailed description.

### BRIEF DESCRIPTION OF THE DRAWINGS

FIG. 1 is a sectional view of the general arrangement of a pacemaker installed in-vivo with a pulse signal generator and lead therefrom inserted in a vein attached to a wall of the heart, and shows scar tissue engaging the lead.

FIG. 2 is an enlarged view of one version of the present disclosure employing a single layer of sheath having the first or distal end thereof secured to the distal end portion of the lead and shows the sheath in place in a view with scar tissue formed thereover.

FIG. 3 is a view showing the initial installation of the one version of FIG. 2 having a single layer of sheath in position as it is attached to the distal end of the lead.

FIG. 4 illustrates the method with a semi-rigid tube assembled over the sheath at the distal end of the lead and extending over and along the free end of the sheath.

FIG. 5 shows the beginning of a fold of the free distal end of sheath tubing over the semi-rigid tube.

FIG. 6 shows the semi-rigid tube moved partially towards the proximal end of the sheath with the end of the sheath held folded over the semi-rigid tube.

FIG. 7 shows the semi-rigid tube in its final position with a length of the sheath fully covering the length of the lead.

FIG. 8 shows the fully assembled sheath on the lead with the semi-rigid tubing removed.

FIG. 9 shows the sheath-and-lead assembly installed in the vein with scar tissue formed thereover.

FIG. 10 shows the initial movement of the lead upon beginning of extraction.

FIG. 11 shows the position of the lead as the lead has been moved from the position of FIG. 10, shown in dashed line, partially toward the proximal end with the sheath peeled away from the scar tissue.

FIG. 12 shows the lead moved from the FIG. 11 position, shown in dashed line, to a position fully extracted from the vein wherein the sheath has been fully peeled from the scar tissue.

FIG. 13 is a cross-sectional view of another version of the lead assembly of the present disclosure in its installed position with a distal end of the sheath secured to the distal end of the lead with a spacer, and secured thereto such as by adhesive bonding.

FIG. 14 shows the initial movement of the sheath exhibiting the Poisson's effect wherein the proximal portion of the sheath where tension is applied to an end of the sheath begins to separate or peel away from the scar tissue.

FIG. 15 shows the sheath continually stretched with the outer surface thereof moved towards the distal end by the Poisson's effect with the outer periphery of the sheath partially separated or peeled away from the scar tissue.

FIG. 16 is a view similar to FIG. 15 showing the stretchable sheath upon continued stretching completely separated or peeled away from the scar tissue by the Poisson's effect and ready to be extracted from the vein.

FIG. 17 is a view showing the lead passing through a wall of the vein.

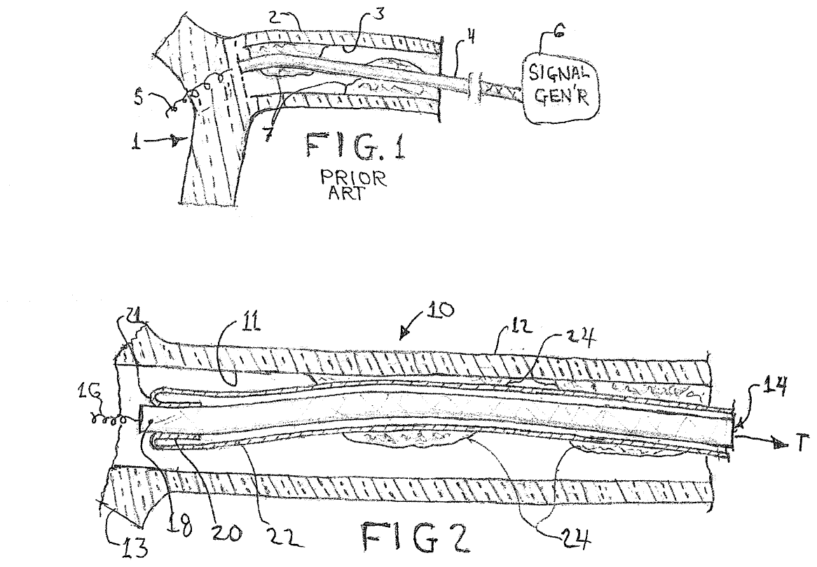

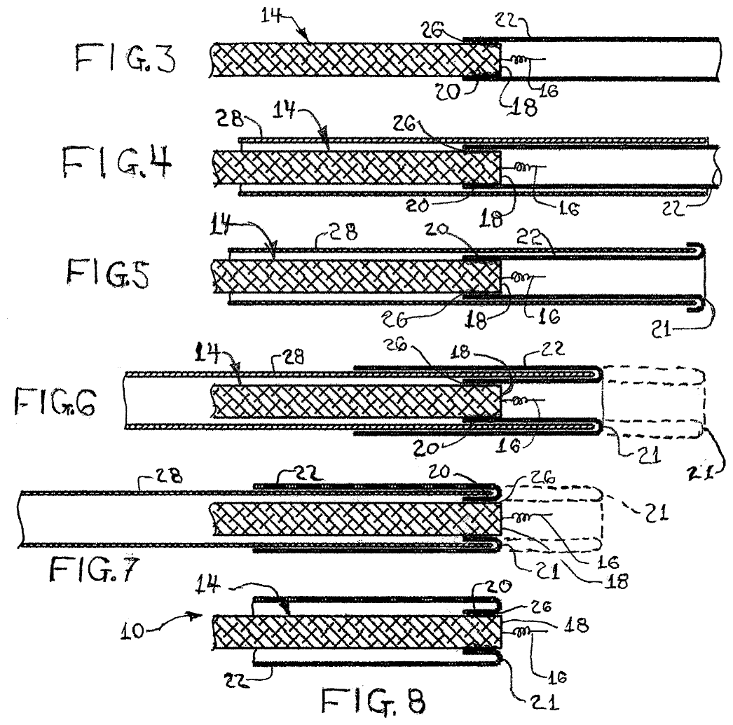

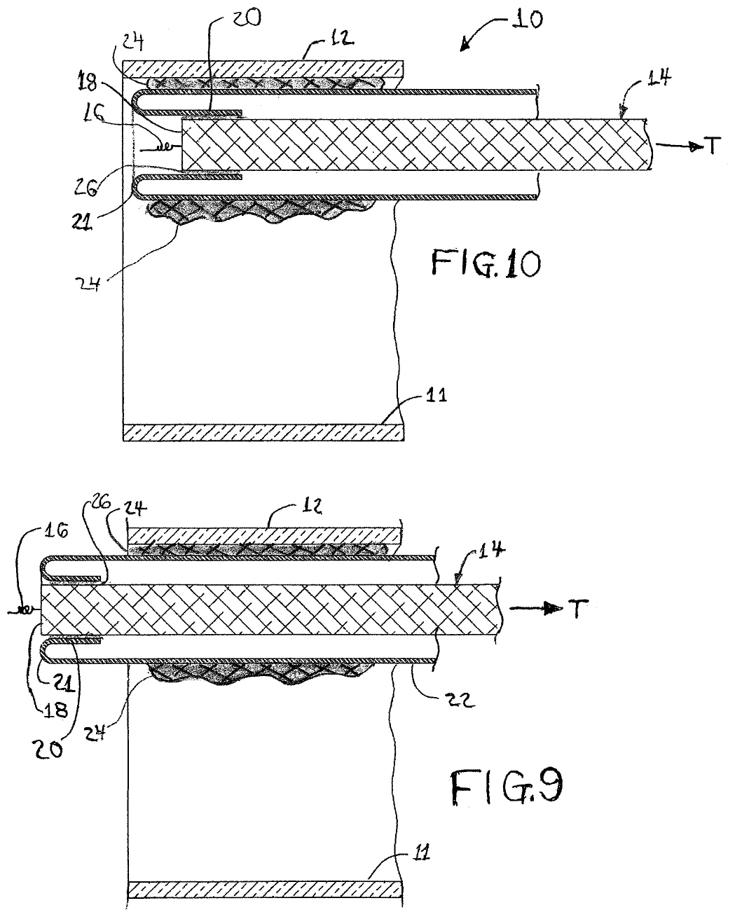

### DETAILED DESCRIPTION

The following description with reference to the accompanying drawings is provided to assist in a comprehensive understanding of one or more versions or embodiments of the present disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the present disclosure. Various exemplary embodiments of the present disclosure are not limited to the specific details of different variations or embodiments and should be construed as including all changes and/or equivalents or substitutes included in the ideas and technological scope of the appended claims. In describing the drawings, where possible similar reference numerals are used for similar elements.

The terms “include” or “may include” used in the present disclosure indicate the presence of disclosed corresponding functions, operations, elements, and the like, and do not limit additional one or more functions, operations, elements, and the like. In addition, it should be understood that the terms “include”, “including”, “have” or “having” used in the present disclosure are to indicate the presence of components, features, numbers, steps, operations, elements, parts, or a combination thereof described in the specification, and do not preclude the presence or addition of one or more other features, numbers, steps, operations, elements, parts, or a combination thereof.

The terms “or” or “at least one of A or/and B” used in the present disclosure include any and all combinations of words enumerated with them. For example, “A or B” or “at least one of A or/and B” mean including A, including B, or including both A and B.

Although the terms such as “first” and “second” used in the present disclosure may modify various elements of the different exemplary embodiments, these terms do not limit the corresponding elements. For example, these terms do not limit an order and/or importance of the corresponding elements, nor do these terms preclude additional elements (e.g., third, fourth, etc.) The terms may be used to distinguish one element from another element. For example, a first mechanical device and a second mechanical device all indicate mechanical devices and may indicate different types of mechanical devices or the same type of mechanical device. For example, a first element may be named a second element without departing from the scope of the various exemplary versions or embodiments of the present disclosure, and similarly, a second element may be named a first element.

It will be understood that, when an element is mentioned as being “connected” or “coupled” to another element, the element may be directly connected or coupled to another element, and there may be an intervening element between the element and another element. To the contrary, it will be understood that, when an element is mentioned as being “directly connected” or “directly coupled” to another element, there is no intervening element between the element and another element.

The terms used in the various exemplary versions or embodiments of the present disclosure are for the purpose of describing specific exemplary versions or embodiments only and are not intended to limit various exemplary versions or embodiments of the present disclosure. As used herein, the singular forms are intended to include the plural forms as well, unless the context clearly indicates otherwise. Use of dimensions, temperatures, ranges, time, relationships (e.g., “perpendicular”, “parallel”), etc. that either use or do not use further adjectives such as “generally”, “substantially”, “about” or “approximately” in the description or claims are intended to cover both the specific dimension, temperature, range, time, relationship, etc., as well as a range of equivalents (function, way, or result) and only intended to be limited by teachings of the prior art.

The terms used in the various exemplary versions or embodiments of the present disclosure are for the purpose of describing specific exemplary version or embodiments only and are not intended to limit various exemplary versions or embodiments of the present disclosure. As used herein, the singular forms are intended to include the plural forms as well, unless the context clearly indicates otherwise.

All of the terms used herein including technical or scientific terms have the same meanings as those generally understood by an ordinarily skilled person in the related art unless they are defined otherwise. The terms defined in a generally used dictionary should be interpreted as having the same meanings as the contextual meanings of the relevant technology and should not be interpreted as having inconsistent or exaggerated meanings unless they are clearly defined in the various exemplary embodiments.

Referring to FIG. 2, one version or embodiment of the present disclosure is shown in an enlarged section view and indicated generally at 10 and has the body passage 11 formed as the interior of the lumen 11 of a vein 12 for which the lumen 11 communicates with the interior of the heart muscle 13. A portion of an electrical signal lead is indicated generally at 14 and has therein electrical conductor wires denoted by reference numeral 16 which are shown as entering into the interior of the heart at 16.

A distal end of the electrical lead 14 as indicated by reference numeral 18 has been secured thereto or attached to a portion 20 of an end of a flexible tubular sleeve or sheath 22; and, the secured portion 20 has a length which comprises a minor portion of the overall length of the sleeve 22. In the present practice, it has been found satisfactory to have the length of the second portion 20 comprise a minor fraction of the overall length of the sheath 22. The remaining unsecured portion of the length of the sheath 22 is then folded back on itself in an annular U-fold 21 and extends over the length of the lead 14. In the present practice it has been found satisfactory to form the sheath 22 of a physiologically biocompatible material which may for example be a polymer, and the sheath 22 preferably has a wall thickness substantially less than the diameter of the sheath. The material employed for sheath 22 must have sufficient flexibility to be assembled over the lead 14 and follow the contour of the lead when secured in vein 12.

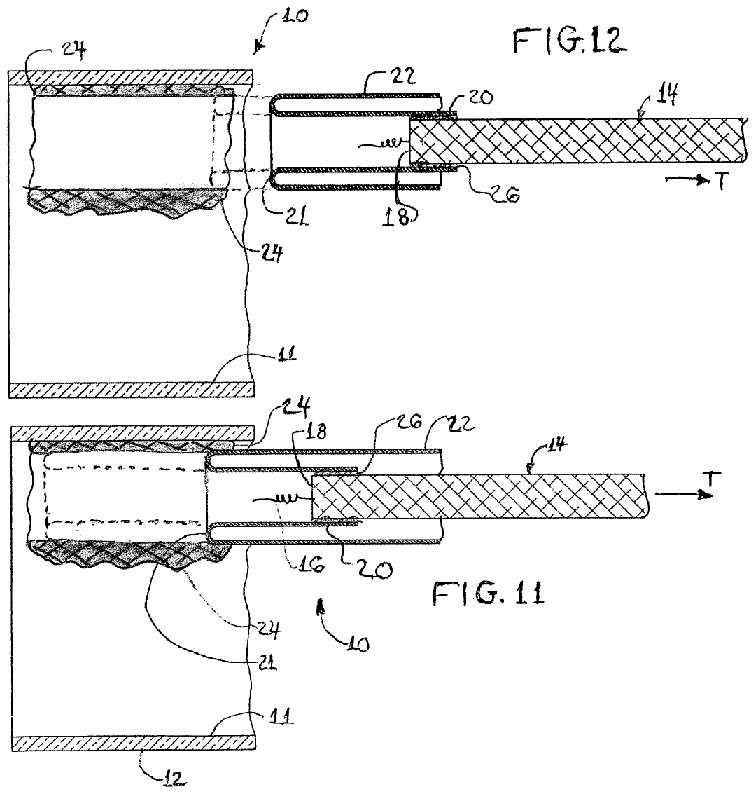

Referring to FIG. 2, the scar tissue is indicated at 24 and is shown as surrounding or partially surrounding a portion or portions of the length of the sheath 22 and thereby attaching those portions to the wall (lumen) 11 of the vein 12. Upon commencing a procedure of the extraction of the lead 14, a tensile force generally indicated by T is applied to the distal end of the sheath 22. Under influence of this tensile force T the lead 14 begins to move towards the right-hand direction of FIG. 2 from its installed position (as also shown enlarged in FIG. 9).

In the present practice, it has been found satisfactory to secure or attach the end portion 20 of the sheath 22 to the distal end 18 of lead 14 by weldment, adhesive bonding, or fasteners such as clamp bands or snap rings. In the present practice weldment comprising sonic welding has been found to be satisfactory. In FIGS. 9, 10, 11, and 12, the sheath 22 is shown secured or attached to the distal end 18 of the lead 14 by adhesive suitable for exposure to blood as indicated at 26. Irrespective of the technique employed for securing end portion 20 of the sheath 22 to distal end 18 of lead 14, the securement must provide a seal of the portion 20 to lead 14 to prevent seepage of blood there-between.

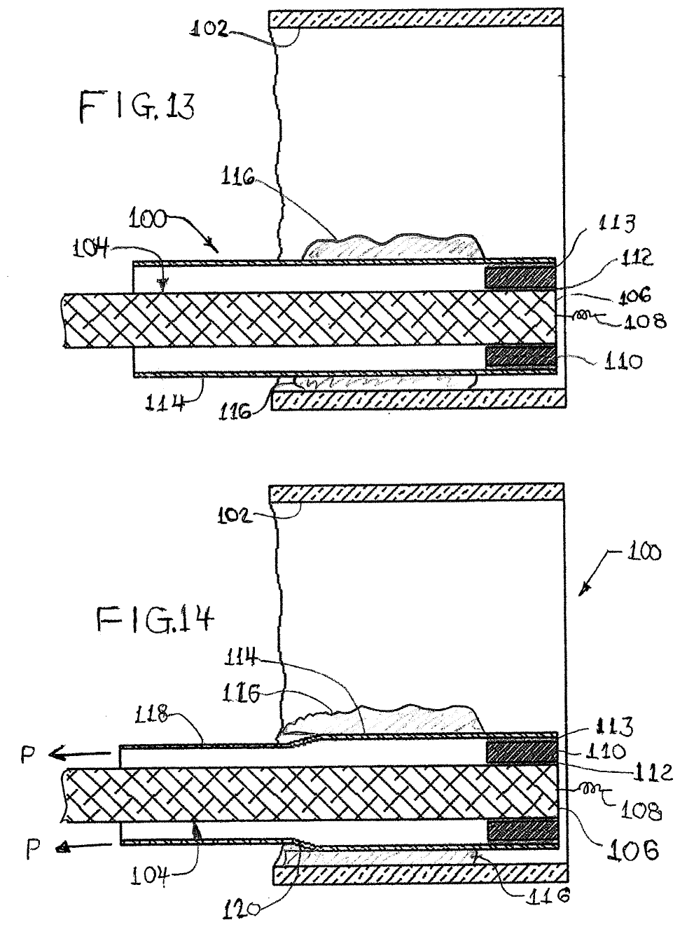

Referring to FIG. 10, distal end 18 of the lead 14 and the secured portion 20 of the sheath have been moved under the influence of the tensile force T to the position from that shown in FIG. 9 to the right-hand position shown in FIG. 10 with reference to FIGS. 2, 9, and 10, the annular U-fold formed between the secured end 20 of sheath 22 and the remaining portion of the sheath is denoted by portion of the sheath is denoted by reference numeral 21. Referring to FIG. 10, the annular U-fold has been moved in a right-hand direction from the position shown in FIG. 9 (which is the installed position) to the position shown in FIG. 10 by the movement of lead 14 under the influence of the tensile force T.

Referring to FIGS. 9 and 10, the lead 14 and sheath 22 are shown in the installed position in FIG. 9; and in FIG. 10 extraction has begun and the annular U-fold 21 has been moved in the right-ward direction from the position shown in FIG. 9 to the position shown in FIG. 10 under the influence of a tensile force indicated by T applied to the lead. Under continued application of the tensile force T the lead and secured portion 20 of the sheath have moved from the position shown in FIG. 10 to the position shown in FIG. 11 with the previous position of FIG. 10 shown in dashed outline in FIG. 11. At this point it will be apparent that the movement of the annular U-fold 11 from the position shown in FIG. 10 to the position shown in FIG. 11 has brought about a peeling action of the sheath's outer surface 22 from the scar tissue 24.

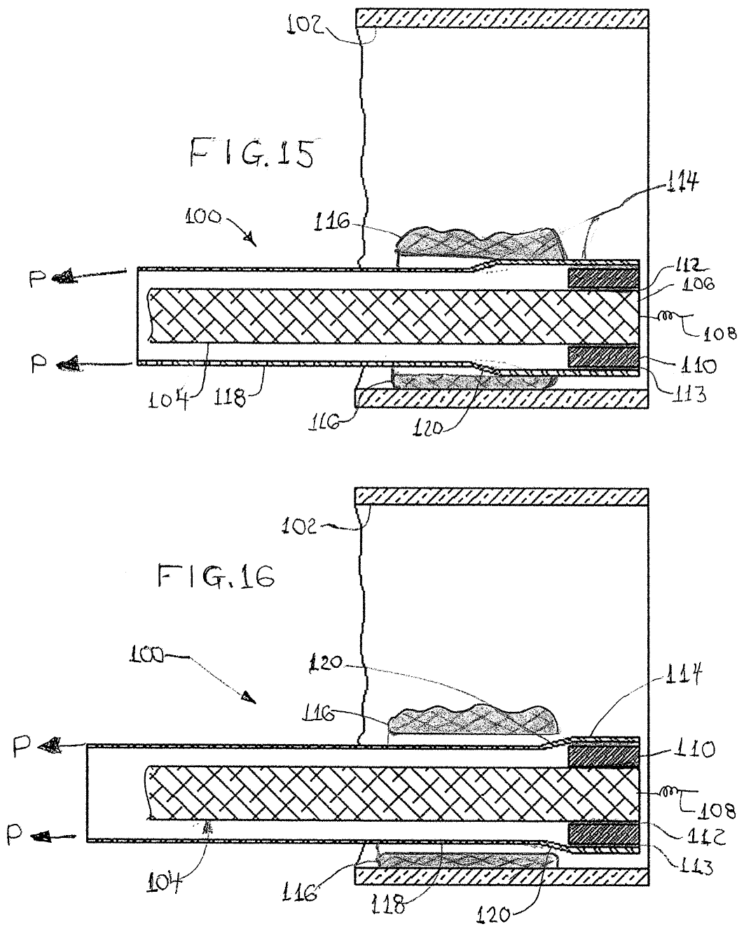

Referring to FIG. 12, the lead 14 under continued application of the tensile force T has moved from the position shown in FIG. 11 outwardly of the vein 12 from the position of FIG. 11 which is shown in dashed outline in FIG. 12 so that the lead is now completely free and extracted from the vein 12 along with the sheath 22. It is also contemplated that the tensile force T could be applied to the sheath 22 to free the sheath and extract the lead from the vein, and as will become more apparent from the version of FIGS. 13-14 to be described further below.

Referring to FIGS. 3 through 8, the method of making the assembly 10 will be described. With particular reference to FIG. 3, a length of tubing for making the sheath 22 is shown as having one end (e.g., a first end) secured such as by adhesive bonding 26, to the distal end 18 of the lead 14 with the remainder of the sheath 22 extending in the opposite direction or away from the distal end 18 of the lead 14. Particularly, an inner surface of the tubing (at or adjacent to the first end of the tubing) is secured to the outer surface of the distal end 18 of the lead 14. Any suitable technique for securing the inner surface of the end of the tubing/sheath 22 to the outer terminal end of the lead 14 can be used such as the aforementioned adhesive bond 28, sonic welding, or clamping bands or snap rings, although these examples should not be construed to limit the present disclosure.

Referring to FIG. 4, a semi-rigid forming tube 28 is received over the lead 14 and the length of the sheath 22. It will be apparent that the semi-rigid forming tube 28 must be of a diameter to closely inter-fit to the outer diameter of the sheath 22. As shown in FIG. 4, the forming tube 28 is preferably axially advanced over the entire length of the sheath 22 and partially over the distal end 18 of the lead (and thus over the secured connection between the sheath and lead).

Referring to FIG. 5, the remote or distal end of the semi-rigid forming tube 28 is positioned and shown as having the furthest or remote distal end of the sheath tube 22 folded in an annular U-fold 21 over the end of the forming tube 28. The elastic nature of the flexible sheath 22 mechanically squeezes an external surface of the forming tube 28, and the U-fold 21 is advanced axially over the tube as the tube is axially advanced (in the left-hand direction as shown in FIGS. 5-7) over the lead 14.

Referring to FIG. 6, the annular U-fold 21 of the material of the sleeve 22 has been pulled left-ward to move the annular U-fold 21 to the position shown in solid line in FIG. 6, with the semi-rigid forming tube 28 having been moved along with the annular fold. The amount of movement to the position shown in FIG. 6 of the annular fold 21 from the position of FIG. 5 is shown in dashed outline in FIG. 6.

Referring to FIG. 7, the material of sheath 22 has been moved further to the position where the annular fold 21 is now coincident with the end of the distal end 18 of the electrical lead 14, i.e., adjacent the secured connection between the terminal ends of the sheath and the lead. The distance moved by the annular fold 21 and the forming tube 28 in FIG. 7 from the position shown in FIG. 6 is indicated in dashed outline in FIG. 7.

Referring to FIG. 8, the forming tube 28 has been withdrawn from the sheath 22 and the lead assembly 14 is in a position to be installed in the body passage. It will be understood that a radial gap between the sheath 22 and the outer surface of the lead is shown in FIG. 8 for ease of illustration, although the sheath will actually radially contract and contact the outer surface of the lead due to the resilient nature of the flexible material of the sheath that seeks to return toward its original, unstretched, reduced diameter. The method disclosed in FIGS. 3 through 8, thus provides a simple and easy-to-prepare method of attaching the sheath 22 with a U-fold 21 to the electrical lead 4 and which may be readily applied upon the original manufacture of the lead assembly for the pacemaker.

Referring to FIGS. 13 and 14, another embodiment of the present disclosure is indicated generally at 100 wherein a portion of the lumen of the vein 102 is shown in an enlarged view in cross-section. An electrical lead generally indicated at 104 is shown disposed within the vein lumen 102 and has the distal end 106 which has electrical conductor 108 shown extending therefrom. An annular radial spacer 110 is optionally secured to distal lead end 106 by adhesive bonding 112. However, it will be understood that the spacer 110 may be secured to the distal end 106 of the lead 104 by one of adhesive bonding, weldment, or fasteners.

A stretchable sheath 114 is received over the lead 104 and the sheath has an end thereof comprising the distal end received over the outer periphery of the spacer 110. The sheath 114 in the embodiment 100 is formed of a stretchable physiologically biocompatible material, which may be a polymeric material and the distal end of the sheath 114 is also secured over the outer periphery of the spacer 110 by adhesive bonding. However, it will be understood that weldment, adhesive bonding, or fasteners may be employed for securing of the sheath 114. The formation of scar tissue is illustrated with reference numeral 116 in FIG. 13. It will be clearly understood that the distal end of the sheath 114 is securely fastened to the distal end 106 of the lead 104 by the bonding 112 between the lead and the spacer, and the bonding 113 between the spacer 110 and sheath such that a seal is provided to prevent any seepage of blood between the lead 104 and sheath 114 irrespective of the technique employed for securing spacer 110 in place.

Referring to FIG. 14, the stretchable sheath 114 is shown as having the tensile force indicated as P applied thereto at its proximal end (left end) in FIG. 14, causing elongation or stretching of the sheath 114 which by Poisson's effect results in reduction in the diameter of the sheath 114 in response to a tensile force applied thereto as indicated at 118.

In the present practice it is deemed satisfactory to form the sheath 114 of material having a stretchability of elongating under a tensile force of 0.25 Newton by an amount of from about 4 mm to 10 mm. At the beginning of the reduction of the sheath diameter due to the Poisson's effect as denoted by reference numeral 120, a separation or peeling of the outer surface of the sheath 114 from the surrounding scar tissue 116 occurs at the scar tissue adjacent the proximal end of lead 104, and then proceeds axially between the sheath and the scar tissue.

It will also be recognized that if the spacer 110 is employed, it may be necessary to use one technique to bond the spacer to the lead 104, and another technique to bond the sheath 114 to the spacer due to the differences in materials. That is, the stretchable sheath 114 may not bond directly to the lead 104 other than by clamping band or snap rings; whereas the spacer 110 could be adhesively bonded to the lead and sheath, or sonic welded to the lead and adhesively bonded to the sheath. If the spacer 110 is not used, the sheath 114 can be optionally attached directly to the lead 104.

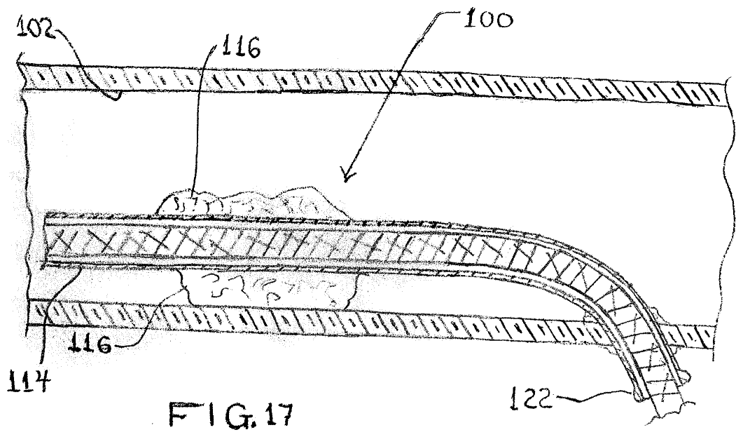

Referring to FIG. 15, continued application of the tensile force P on the stretchable sheath 114 has resulted in an increased length of the reduced diameter 118 by virtue of movement of the point separation or of peeling 120 towards the distal end of the lead in a rightward direction as is shown in FIG. 15 as having moved further towards the distal end of the lead 104.

Referring to FIG. 16, continued application of tensile force P on the proximal end of the stretchable tubing 114 has caused the point of separation or peeling 120 to move or propagate away from the proximal end to the distal end of the lead 104. This completely separates or peels the outer surface of the sheath 114 from the scar tissue 116 thus enabling the sheath 114 and lead 104 to be extracted easily from the scar tissue. If desired, the sheath 114 may have a raised surface (not shown) at the proximal end for facilitating gripping for stretching.

FIG. 17 illustrates how the lead with sheath 114 is partially encompassed by scar tissue 116 and exits through a wall of the vein.

The present disclosure thus describes a lead assembly for in vivo insertion into a body passage such as the lumen of a vein and is particularly applicable for assembly of a cardiac pacemaker. The disclosure describes one embodiment wherein the lead has a distal end thereof provided with an end portion of a flexible sheath attached thereto with the sheath initially extending outwardly from the distal end and subsequently folded in an annular U-fold over the lead to provide a sheath along the length from the distal end to the proximal end. By application of a tensile force to the proximal end of the lead, the annular fold causes the material of the sheath to peel away from any surrounding scar tissue as the lead is pulled out towards the proximal end and thus provide for easy nondestructive extraction of the lead from the vein. In another embodiment, a single layer flexible sheath is attached at the distal end of a lead; and, upon application of a tensile force to the stretchable sheath, the Poisson effect causes progressive reduction in diameter of the sheath, that in turn, causes separation or peeling of the outer surface of the sheath, initiating from the proximal end and progressing to the distal end of the sheath thereby freeing the lead from surrounding scar tissue and enabling easy extraction of a lead from the scar tissue and the vein. A method of assembling the one embodiment is described where a semi-rigid forming tube is used as a temporary tool over the lead in order to form an initial annular fold on the distal end of the sheath.

This written description uses examples to describe the disclosure, including the best mode, and also to enable any person skilled in the art to make and use the disclosure. Other examples that occur to those skilled in the art are intended to be within the scope of the invention if they have structural elements that do not differ from the same concept or that do not differ from the literal language of the claims, or if they include equivalent structural elements with insubstantial differences from the same concept or from the literal language of the claims. Moreover, this disclosure is intended to seek protection for a combination of components and/or steps and a combination of claims as originally presented for examination, as well as seek potential protection for other combinations of components and/or steps and combinations of claims during prosecution.

Although specific advantages have been enumerated above, various embodiments may include some, none, or all of the enumerated advantages. Although exemplary embodiments are illustrated in the figures and description herein, the principles of the present disclosure may be implemented using any number of techniques, whether currently known or not. Moreover, the operations of the systems and apparatuses disclosed herein may be performed by more, fewer, or other components, and the methods described herein may include more, fewer, or other steps. Additionally, steps may be performed in any suitable order.

To aid the Patent Office and any readers of this application and any resulting patent in interpreting the claims appended hereto, applicants do not intend any of the appended claims or claim elements to invoke 35 USC 112 (f) unless the words “means for” or “step for” are explicitly used in the particular claim.

What is claimed is:

1. An electrical lead assembly received in an associated body passage, the lead assembly comprising:
a) an electrical signal lead sized for receipt in the associated body passage, the lead having a proximal end and a distal end;
b) a sheath having an outer surface configured and sized for receipt in the associated body passage and an inner surface received over the lead, the sheath having a first end adjacent the distal end of the lead and extending continuously to a second end adjacent the proximal end of the lead with the first end of the sheath having a portion secured to the lead adjacent the distal end thereof wherein the secured portion includes an annular fold of the sheath thereby forming the secured portion of the first end of the sheath.

2. The lead assembly of claim 1, wherein the sheath and the secured portion of the first end thereof are formed integrally as a one-piece member.

3. The lead assembly of claim 1, wherein the lead assembly is removable from the associated body passage by application of a tensile force to the proximal end of the lead whereupon the annular fold moves progressively towards the proximal end separating the outer surface of the sheath and enabling the lead and sheath to be removed from the associated body passage.

4. The lead assembly of claim 1, wherein the secured portion of the first end of the sheath is attached to the lead by one of (i) adhesive, (ii) weldment, and (iii) mechanical fastening.

5. The lead assembly of claim 1, wherein the sheath is formed of a physiologically biocompatible material.

6. The lead assembly of claim 1, wherein the sheath is formed of a polymeric material.

7. The lead assembly of claim 1, wherein the sheath is formed of a flexible polymeric material.

8. The lead assembly of claim 1, wherein the secured portion of the first end of the sheath is attached to the lead by sonic welding.

9. The lead assembly of claim 1, wherein the secured portion of the first end of the sheath is attached to the lead by one of clamp bands and snap rings.

10. The lead assembly of claim 1, wherein, the secured portion of the first end of the sheath is attached to the lead by annular physiologically biocompatible adhesive.

11. The lead assembly of claim 1, wherein the sheath is formed as an integral one-piece continuous member.

12. The lead assembly of claim 1, wherein the assembly is removable from the associated body passage by application of a tensile force to the proximal end of the lead whereupon the annular fold moves progressively towards the proximal end of the lead and the lead and sheath are extracted from the associated body passage.

13. The lead assembly of claim 1 wherein the annular fold of the sheath extends inwardly from an outer surface of the sheath to form the secured portion of the first end of the sheath.

14. An electrical lead assembly received in vivo in an associated body passage, the lead assembly comprising:
a) an electrically conductive lead sized for receipt in the associated body passage, the lead having an outer surface extending between a proximal end and a distal end; and,
b) an integrated feature that facilitates extraction of the lead from the associated body passage, wherein the integrated feature that facilitates the extraction includes a sheath sized for receipt in the associate body passage and received over the lead, the sheath including:
i. a first segment extending from adjacent the distal end of the lead the first segment having a surface secured to the outer surface of the lead and extending towards the proximal end for a portion of the outer surface of the lead, the first segment having a second surface facing radially outward from the lead outer surface, and
ii. a second segment extending from adjacent the proximal end to adjacent the distal end of the lead, the second segment having an inner surface received over the first segment and the outer surface of the lead and the second segment further having an outer second surface abutting with an inner surface of the associated body passage that receive the lead therein wherein the first segment is connected to the second segment.

15. The lead assembly of claim 14, wherein the first surface of the first segment of the sheath is secured to the outer surface by one of (i) weldment, (ii) mechanical fastening, and (iii) adhesive bonding.

16. The lead assembly of claim 14, wherein the first segment is connected to the second segment with an annular fold.

17. The lead assembly of claim 14, wherein the first and second segments are formed integrally with an annular fold.

18. The lead assembly of claim 17, wherein the one piece member comprises a flexible tube.

19. The lead assembly of claim 14, wherein the first and second segments of the sheath are formed of flexible material.

20. The lead assembly of claim 14, wherein the first and second segments of the sheath are formed of physiologically biocompatible flexible material.

21. The lead assembly of claim 14, wherein the first and second segments of the sheath are formed of a one-piece tubular member of flexible polymeric material.

22. The lead assembly of claim 14, wherein upon application of a tensile force to the proximal end of the lead, the first segment of the sheath and lead move toward the proximal end permitting extraction of the sheath and lead from the associated body passage.

23. An electrical lead assembly received in vivo in an associated body passage, the lead assembly comprising:
a) an electrical signal lead sized for receipt in the associated body passage, the lead having a proximal end and a distal end, and
b) a tubular sheath member having a proximal end and a distal end and received in the associated body passage and an inner surface received over the lead with the distal end of the sheath secured to the distal end of the lead wherein the sheath is formed of a stretch-able material.

24. The lead assembly of claim 23, wherein the sheath is formed of elastic material.

25. The lead assembly of claim 23, wherein upon the sheath being gripped by tissue formation in the associated body passage, upon application of a tensile force to the proximal end of the sheath, elastic stretching of the sheath occurs, effecting progressive separation from the end of the sheath from the associate body passage progressively from the proximal end towards the distal end thereof and enabling extraction of the sheath and lead from the associated body passage.

26. The lead assembly of claim 24, wherein the sheath is formed of polymeric material.

27. The lead assembly of claim 24, wherein the sheath is formed of physiologically biocompatible material.
